(19) United States
(12) Patent Application Publication
(10) Pub. No.: US 2022/0152222 A1
(43) Pub. Date: May 19, 2022

(54) Gene Therapy for Addiction Disorders

(71) Applicant: PATHWAYS NEURO PHARMA, INC., Tomball, TX (US)

(72) Inventors: Warren C. LAU, Houston, TX (US); Brad THOMPSON, Calgary (CA)

(73) Assignee: PATHWAYS NEURO PHARMA, INC., Tomball, TX (US)

(21) Appl. No.: 17/434,350

(22) PCT Filed: Feb. 27, 2020
(86) PCT No.: PCT/US2020/020216

§ 371 Date: Aug. 26, 2021

Related U.S. Application Data
(60) Provisional application No. 62/811,339, filed on Feb. 27, 2019.

(51) Int. Cl. A61K 48/00

(52) U.S. Cl. CPC A61K 48/005; A61K 48/0075

(57) ABSTRACT

The present invention encompasses treatments for neurologic disorders with recombinant vims vectors encoding G-protein coupled receptors. In particular, the invention is directed to the treatment of addiction disorders including but not limited to alcohol addiction and opiate addiction.

### CROSS-REFERENCE TO RELATED APPLICATIONS

This application claims the benefit of U.S. Provisional Patent Application Ser. No. 62/811,339, filed Feb. 27, 2019, the full disclosure of which is incorporated herein by reference.

### INCORPORATION BY REFERENCE OF A SEQUENCE LISTING PROVIDED AS A TEXT FILE

A sequence listing is provided herewith as a text file, “101907-5001-WO-Sequence-Listing.txt” created on Feb. 27, 2020 and having a size of 11,000 bytes. The contents of the text file are incorporated herein by reference in their entirety.

### FIELD OF THE INVENTION

The present invention generally relates to gene therapy for neurological disorders, including addiction disorders including opiate and alcohol addiction.

### BACKGROUND OF THE INVENTION

Neuroreceptors, in particular, G protein coupled receptors (GPCRs), play an important role in many mental functions. GPCRs represent one of the most common targets for drug discovery. GPCRs primarily expressed in central nervous system tissues represent attractive targets for advanced gene therapies for the treatment of complex behavioral pathologies, including but not limited to alcoholism, opiate addiction, depression anxiety, and others.

Serotonin receptors such as the 5-Hydroxytryptamine receptor, HTR4 (SEQ ID NO: 1), are GPCRs that occur in the brain, primarily within the putamen, caudate nucleus, nucleus accumbens, globus pallidus, and the substantia nigra. HTR4 agonists, such as zacopride, are known to have potent anti-anxiety and cognitive enhancing effects.

Dopamine receptors, another class of GPCRs, are key mediators of several important brain functions and play key roles in muscular motor control. Dopamine receptor D1, D1(A) (SEQ ID NO: 2), agonists such as doxanthrine are useful in the treatment of Parkinson's disease and have shown efficacy in improving overall mood and sense of well-being in patients suffering from anxiety. D1(A) is primarily expressed in the dorsal and ventral striatum and to a lesser degree in the amygdala, cerebral cortex, and hypothalamus.

Orphan GPCRs, those GPCRs whose natural ligand(s) remain undiscovered, are especially attractive targets for pharmaceutical development. One highly conserved GPCR, GPR139 (SEQ ID NO: 3), has recently been shown to play a key role in brain function, and is highly expressed in regions of the brain (circum-ventricular regions of the habenula and septum and the interpeduncular nucleus) are associated with addiction, anxiety, and mood regulation. An association between GP139 expression in the habenula and alcoholism has been demonstrated in a rat model. This model suggests that administration of GPR139 agonists reduced addictive alcohol consumption may reduce other compulsive addiction-like behaviors.

Alcoholism and opiate addiction are increasing in the United States. However, treatment with drugs is limited and behavioral therapy while effective over the short term has very high rates of relapse.

### SUMMARY OF THE INVENTION

The present invention relates to a method for inducing endogenous production and/or over production of a neuroreceptor and/or sub-peptides of a neuroreceptor in certain areas of the brain utilizing gene transfer vectors such as lentivirus vectors, adeno associated virus and other gene transfer vectors comprising nucleic acids or encoding one or more neuroreceptors and/or ligand binding sub-peptides of a neuroreceptor.

The present invention is also directed to methods for increasing expression of GPCR receptors of the brain and in particular the putamen, caudate nucleus, nucleus accumbens, globus pallidus, and/or the substantia nigra and habenula and circumventricular regions of the habenula. In a preferred embodiment the gene vector increases expression of HTR4 in various regions of the brain.

Other regions of the brain which may be targeted by the gene transfer vectors of the present invention include the dorsal and/or ventral striatum, the amygdala, cerebral cortex, and/or the hypothalamus. In a preferred embodiment the vector increases the expression of D1(A) in the CNS.

Other aspects of the present invention relate to gene transfer vectors targeted to the habenula and circumventricular regions of the habenula. In some embodiments the gene vector targets specific to the habenula region and introduces into the habenula genes encoding neuroreceptors. In a preferred embodiment the gene vector upregulates expression or supplements the expression of endogenous of GPR139.

Other embodiments of the present invention relate to a pharmaceutical composition comprising gene vectors that further comprise the gene transfer vectors described above in a pharmaceutically acceptable carrier, and/or an excipient that upregulates or supplements the production of a neuroreceptor and/or a sub-peptide of a neuroreceptor.

Some embodiments of the present invention relate to methods and compositions for enhancing delivery of the gene transfer vector to target tissues within the brain. Such methods and compositions may include, without limitation, osmotic, ultrasound, low dose radiation, or administration of bradykinin to disrupt the blood brain barrier to allow access of the gene transfer vector to the target. Other methods for circumventing the blood brain barrier may include, without limitation, methods and compositions comprising viral mediated delivery, use of surgically implanted catheters, and use of liposomal carriers, intrathecal, and intracranial injection.

Some embodiments of the present invention include a kit used for treatment of a condition, or for delivery of a therapy to a subject. The kit comprises a unit dosage of a gene transfer vectors as described herein, a carrier for the unit dosage, and instructions for administering the unit dosage to the subject. The gene vector may increase the production of a neuroreceptor and/or a sub-peptide of a neuroreceptor in a target tissue. The carrier may be a pharmaceutically acceptable carrier as commonly understood in the art. The instructions may describe how the solid carrier may be administered to a subject for an optimal effect. The instructions may also describe how the liquid carrier may be administered to a subject by various routes of administration.

Some embodiments of the present invention relate to methods of treating certain neurologic disorders. The method comprises a step of administering to a subject a therapeutically effective amount of a gene vector that upregulates a production of a neuroreceptor and/or a sub-peptide of a neuroreceptor. Disorders include behavioral disorders such as anxiety, alcoholism, opiate addiction, Post-Traumatic Stress Disorder (PTSD), and other disorders set forth herein.

Another embodiment of the invention include gene transfer vectors capable of introducing nucleic acids encoding GPCRs into neural tissues: gammaretroviruses, lentivirus, flavivirus, influenza, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, herpes simplex virus, varicellovirus, cytomegalovirus, or combinations thereof. A preferred embodiment of the invention comprises a viral vector such as adeno-associated viruses covering adenoviruses vectors herpes virus vectors, measle viruses, vaccinia viruses, retroviral vectors including lentiviral vectors and other virus vectors known in the art. The viral vectors of the present invention may be collectively referred to as recombinant virus vectors (RVV).

Without being bound by any particular theory, embodiments of the present invention may be useful for treating conditions including, but not limited to, addiction, such as alcohol and/or opiate addition, depression, psychosis, Post-Traumatic Stress Disorder (PTSD), Alzheimer's disease, and Parkinson's disease.

### DESCRIPTION OF THE DRAWINGS

FIG. 1 sets out SEQ ID NO: 1 which represents the sequence of a nucleic acid encoding HT4.

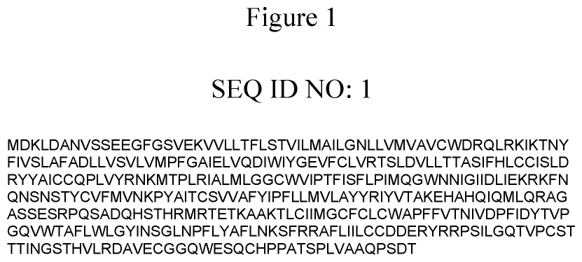

FIG. 2 sets out SEQ ID NO: 2 which sets out the sequence of a nucleic acid encoding D1(CA).

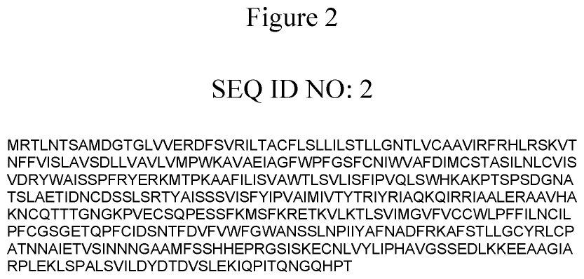

FIG. 3 sets out SEQ ID NO: 3 which represents the sequence of a nucleic acid encoding GPR139.

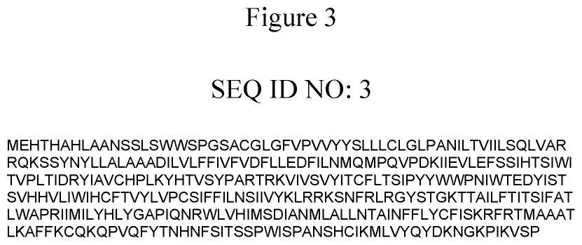

### DETAILED DESCRIPTION OF THE INVENTION

The present invention relates to one or more gene vectors (e.g., gene transfer vectors (i.e., recombinant virus vectors (RVV)), therapies, treatments and methods of use of the vectors and/or therapies and/or treatments for increasing the production of a neuroreceptor and/or a sub-peptide of a neuroreceptor.

### Definitions

Unless defined otherwise, all technical and scientific terms used herein have the meanings that would be commonly understood by one of skill in the art in the context of the present specification. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, of a given value or range. It is understood that such a variation is always included in any given value provided herein, whether or not it is specifically referred to.

As used herein, the term “gene vector” refers to gene transfer vector comprising a nucleic acid encoding GPCR that, when administered to a patient increases GPCR expression in the relevant part of the brain thereby ameliorating certain disorders of the brain including but not limited to addictive behaviors, physical reactions, and/or one or more physiologic reactions in the patient.

As used herein, the term “excipient” refers to any substance, not itself a gene vector, which may be used as a component within a pharmaceutical composition or a medicament for administration of a therapeutically effective amount of the gene vector to a subject. Additionally, or alternatively, an excipient may alone, or in combination with further chemical components, improve the handling and/or storage properties and/or to permit or facilitate formation of a dose unit of the gene vector. Excipients include, but are not limited to, one or more of: a binder, a disintegrant, a diluent, a buffer, a solvent, a thickening gene vector, a gelling agent, a penetration enhancer, a solubilizing agent, a wetting agent, an antioxidant, a preservative, a surface active agent, a lubricant, an emollient, a substance added to improve the appearance or texture of the composition, and a substance used to form the pharmaceutical compositions or medicaments. Any such excipients can be used in any dosage forms according to the present invention. The foregoing classes of excipients are not meant to be exhaustive but are provided merely as illustrative of what a person of skill in the art would know; a person of skill in the art would also recognize that additional types and combinations of excipients may be used to achieve delivery of a therapeutically effective amount of the gene vector to a subject through one or more routes of administration.

As used herein, the term “subject” refers to any therapeutic target that receives the gene transfer vectors described herein. The subject can be a vertebrate, for example, a mammal including a human. The term “subject” does not denote a particular age or sex. The term “subject” also refers to one or more cells of an organism; an in vitro culture of one or more tissue types, an in vitro culture of one or more cell types; ex vivo preparations; and a sample of biological materials such as tissue and/or biological fluids.

As used herein, the term “medicament” refers to a medicine and/or pharmaceutical composition that comprises the gene vector and that can promote recovery from a disease, disorder or symptom thereof, and/or that can prevent a disease, disorder or symptom thereof, and/or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition for administration of the gene vector to a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the gene vector. Some examples of pharmaceutically acceptable carriers and their formulations are described in Remington (1995, The Science and Practice of Pharmacy (19th ed.) ed. A. R. Gennaro, Mack Publishing Company, Easton, Pa.), the invention of which is incorporated herein by reference. Typically, an appropriate amount of a pharmaceutically acceptable carrier is used in the formulation to render the formulation isotonic. Examples of suitable pharmaceutically acceptable carriers include, but are not limited to: saline solutions, glycerol solutions, ethanol, N-(1(2,3-dioleyloxy) propyl)-N,N,N-trimethylammonium chloride (DOTMA), diolesylphosphotidylethanolamine (DOPE), and liposomes of various constituents. Such pharmaceutical compositions contain a therapeutically effective amount of the gene transfer vector, together with a suitable amount of one or more pharmaceutically acceptable carriers and/or excipients so as to provide a form suitable for proper administration to the subject. The formulation should suit the route of administration. For example, oral administration may require that the formulation incorporate enteric coatings to protect the gene vector from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the terms “production”, “producing”, and “produce” refer to the synthesis and/or replication of DNA, the transcription of one or more sequences of RNA, the translation of one or more amino acid sequences, the post-translational modifications of amino acid sequences, and/or the production or functionality of one or more regulatory molecules that can influence the production or functionality of an effector molecule.

As used herein, the terms “promote”, “promotion”, and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence of and/or the progression of the disease or disorder within the subject.

As used herein, the term “target cell” refers to one or more cells that are deleteriously affected, either directly or indirectly, by a condition.

As used herein, the terms “treat”, “treatment”, and “treating” refer to obtaining a desired pharmacologic and/or physiologic effect. The effect may be prophylactic in terms of completely or partially preventing an occurrence of a disease, disorder or symptom thereof, and/or may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and (c) ameliorating the disease.

As used herein, the term “therapeutically effective amount” refers to the amount of the gene vector used that is of sufficient quantity to ameliorate, treat and/or inhibit one or more of a disease, disorder or a symptom thereof. The “therapeutically effective amount” will vary depending on the gene vector used, the route of administration of the gene vector, and the severity of the disease, disorder or symptom thereof. The subject's age, weight, and genetic make-up may also influence the amount of the gene vector that will be a therapeutically effective amount.

As used herein, the terms “unit dosage form” and “unit dose” refer to a physically discrete unit that is suitable as a unitary dose for patients. Each unit contains a predetermined quantity of the gene vector and optionally, one or more suitable pharmaceutically acceptable carriers, one or more excipients, one or more additional active-ingredients, or combinations thereof. The amount of gene vector within each unit is a therapeutically effective amount.

As used herein, the term “upregulate” refers to increasing expression of a particular gene product, for example, GPCRs in the brain. Means for upregulation may include, without limitation, increasing transcription, translation or improving stability or activity of the target gene product. Upregulation may also comprise inactivation or decreasing expression or efficacy of negative regulatory proteins or other factors to increase expression of the target gene product. In some embodiments, upregulation comprises providing one or more copies of the gene encoding the target gene product. The gene encoding the target gene product may comprise one or more heterologous promoters and may further comprise mutant forms of the gene to increase expression or enzymatic activity of the target gene product. In other embodiments, upregulation of the target gene product may comprise selection for mutations that provide increased expression or enzymatic activity of the target gene product.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also, encompassed within the invention, subject to any specifically excluded limit in the stated range. Where the stated range includes one or both of the limits, ranges excluding either or both of those included limits are also included in the invention.

Some embodiments of the present invention, the invention encompass administering the gene vector to the subject by an intravenous route, an intramuscular route, an intraperitoneal route, an intrathecal route, an intravesicular route, an intraventricular route, a topical route, an intranasal route, a transmucosal route, a pulmonary route, an intravenous route. The gene vector may be administered directly to the central nervous system using stereotactic and other neurosurgical means.

In some embodiments of the present invention, the gene vector includes viral vectors further comprising cargo nucleic acid encoding a GPCR such as HT4, DI(A) or GPR139, used for the introduction of nucleic acids encoding GPCRs and/or subpeptides of GPCRs to targets in the central nervous system.

In some embodiments of the present invention, the gene transfer is a virus selected from the group comprising or consisting of: gammaretroviruses, lentivirus, flavivirus, influenza, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, herpes simplex virus, varicellovirus, cytomegalovirus, or combinations thereof. In preferred embodiments, the gene transfer vector is an adeno-associated virus lentivirus, herpes viruses, and other viruses that may be neurotropic.

The present invention is also directed to treating a condition such as alcoholism and/or opiate addition by administering a therapeutically effective amount of gene transfer vector and its GPCR encoding nucleic acid. In some embodiments of the present invention, the therapeutically effective amount of the vector that is administered to a patient is between about 10 and about 1×10¹⁶ TCID50/kg (50% tissue culture infective dose per kilogram of the patient's body weight). In some embodiments of the present invention, the therapeutically effective amount of the gene vector that is administered to the patient is about 1×10¹³ TCID50/kg. In some embodiments of the present invention, the therapeutically effective amount of the gene vector that is administered to a patient is measured in TPC/kg (total particle count of the gene vector per kilogram of the patient's body weight). In some embodiments, the therapeutically effective amount of the gene vector is between about 10 and about 1×10¹⁶ TPC/kg.

Some embodiments of the present invention relate to a method of treating a condition wherein the method comprises a step of administering to the subject a therapeutically effective amount of a gene transfer vector and its cargo nucleic acid that will upregulate production of a neuroreceptor and/or a sub-peptide of a neuroreceptor, preferably but not limited to the habenula and circumventricular areas of the habenula.

In some embodiments, the invention comprises a method for treating a patient with a disorder such as addiction, anxiety, and/or depression with a gene transfer vector comprising HT4 (SEQ ID NO: 1) into a viral vector. Exposure of a therapeutically effective dose of the vector to cells within the target tissue, such as the putamen, caudate nucleus, nucleus accumbens, globus pallidus and/or the substantia nigra habenula and circumventricular regions of the habenula, increases expression of HT4 within the target tissue thereby ameliorating or improving the symptoms of the disorder.

In some embodiments the invention comprises a method for treating a patient with a disorder such as addiction, anxiety, and/or depression with a gene vector comprising D1(A) (SEQ ID NO: 2) introduced into a viral vector. Exposure of a therapeutically effective dose of the gene transfer vector to cells within the target tissue, such as the dorsal and/or ventral striatum, the amygdala, cerebral cortex and/or hypothalamus will increase expression of D1(A) within the target tissue thereby ameliorating or improving the symptoms of the disorder.

In some embodiments the invention comprises a method for treating a patient with a disorder such as addiction, anxiety, and/or depression with a gene transfer vector comprising GPR139 (SEQ ID NO: 3) introduced into a viral vector. Exposure of a therapeutically effective dose of the vector to cells within the target tissue, such as the habenula circumvent and/or the interpeduncular nucleus will increase expression of GPR139 within the target tissue thereby ameliorating or improving the symptoms of the disorder, i.e., reduce craving for alcohol and/or opiates.

### EXAMPLES

The invention is further described by reference to the following examples, which are provided for illustration only. The invention is not limited to the examples but rather includes all variations that are provided by the teachings provided herein.

### Example 1

### Upregulation and Therapeutic Effect of HT4 Expression

Adeno associated virus vector genome (e.g., AAV-20 or other well-known AAV-vectors) plasmids comprising codon optimized nucleic acid encoding HT4 (SEQ ID NO: 1), a CASI promoter and a SV40 polyA signal positioned between AAV2 inverted terminal repeats are produced by cotransfection of human embryonic kidney 293 cells with the AAV-genome and packaging plasmids and are purified as described by Halbert, et al., [Methods Mol. Biol. 1687:257-66 (2018)]. The titers of the resulting purified viral preparations (AAV-HT4) are determined by quantitative polymerase chain reaction analysis.

Intramuscular, intracranial, intrathermal, intravenous administration of approximately 1×10¹¹ infectious particles of AAV-HT4 into subject mice (BALB/c obtained from Charles River Laboratories, Wilmington, Mass.) will produce mice with increased levels of HT4 expression in the putamen, caudate nucleus, nucleus accumbens, globus pallidus, and the substantia nigra. The level of upregulated HT4 expression may be determined by quantitative fluorescence utilizing anti-HT4 antibodies (e.g., Cat. No. NLS656, Novus Biologicals, Centennial, Colo.; Cat. No. S0195, Millipore-Sigma, St. Louis, Mo.). Increased levels of upregulation of HT4 may be observed in mice that have undergone low dose radiation exposure to the relevant brain tissues.

The effect of upregulation of HT4 on anxiety levels of a subject animal is determined by use of a series of commonly used tests such as the Open Field test, the Elevated Plus Maze test, or the Dark/Light Box test described by Holmes [Neurosci. Biobehay. Rev. 25(3):261-73 (2001)] (it should also be noted that anxiety is a common symptom associated with alcohol and opiate withdrawal). A statistically significant difference in anxiety observed between animals treated with AAV-HT4 indicates that the disclosed compositions and methods provide anxiolytic therapy.

### Example 2

### Upregulation and Therapeutic Effect of D1(A) Expression

A codon optimized nucleic acid sequence encoding D1(A) (SEQ ID NO: 2) is inserted into an MSCV vector originating from a murine stem-cell virus between the vector IRES sequence and the 3′-LTR (Retro-D1(A)). Expression of the D1(A) sequence in Retro-D1(A) is driven from a highly active promoter within the upstream 5′-LTR. The MSCV vector and packaging cell lines are obtained from Takara (Cat. No. 634401, Takara Bio USA, Mountain View, Calif.) and used as described by the manufacturer to purify high titers of Retro-D1(A).

Intravenous administration of approximately 1×10¹² Retro-D1(A) into subject mice (BALB/c obtained from Charles River Laboratories, Wilmington, Mass.) produces mice with increased levels of D1(A) expression in the dorsal and/or ventral striatum, the amygdala, cerebral cortex and/or hypothalamus. The level of upregulated DI(CA) expression may be determined by quantitative fluorescence utilizing anti-D1(A) antibodies (e.g., Cat. No. SG2-D1a, Novus Biologicals, Centennial, Colo.; Cat. No. AB9141, Millipore-Sigma, St. Louis, Mo.). Increased levels of upregulation of D1(A) may be observed in mice that have undergone focused low-intensity ultrasound disruption of the blood brain barrier.

As with HT4, the effect of upregulation of D1(A) on anxiety levels of a subject animal may be determined by use of a series of commonly used tests such as the Open Field test, the Elevated Plus Maze test, or the Dark/Light Box test described by Holmes [Neurosci. Biobehay. Rev. 25(3):261-73 (2001)]. A statistically significant difference in anxiety observed between animals treated with AAV-D1(A) indicates that the disclosed compositions and methods provide anxiolytic therapy.

### Example 3

### Upregulation and Therapeutic Effect of GPR139

A codon optimized nucleic acid sequence encoding GPR139 (SEQ ID NO: 3) is inserted into the multiple cloning site pLVX-IRES-mCherry vector (lentivirus vector). The vector and packaging cell lines are obtained from Takara (Cat. No. 632182, Takara Bio USA, Mountain View, Calif.) and used as described by the manufacturer to purify high titers of the desired gene vector (LVX-GPR139).

Stereotactic introduction of approximately 1×10¹¹ LVX-GPR139 into the habenula of subject rats (Wistar rats available from Charles River Laboratories, Wilmington, Mass.) will produce rats with increased levels of GPR139 expression in habenula (the neuroanatomical site associated with addictive behaviors). The level of upregulated HT4 expression may be determined by quantitative fluorescence utilizing anti-GPR139 antibodies (e.g., Cat. No. NLS2717, Novus Biologicals, Centennial, Colo.; Cat. No. SAB4500335, Millipore-Sigma, St. Louis, Mo.). The Lux-GPR139 vector may also be introduced to the habenula by various methods detailed above for temporarant disrupting the blood brain barrier before or after intravenous administration, or by any of the methods discussed above.

A cohort of subject rats exhibiting compulsive alcohol self-administration behavior as described by Kononoff, et al., [eNeuro 2018; 10.1523/ENEURO.0153-16.2018] is treated with LVX-GPR139 by the procedure described above. The predicted effect of upregulation of GPR139 on such rats is to decrease the frequency of alcohol self-administration events. A statistically significant decrease in self-administration of alcohol in the rats treated with LVX-GPR139 versus those treated with vector alone indicates that the disclosed compositions and methods provide a therapy for addictive behaviors.

Similarly, GPR139 may be introduced into the cloning sites of AAV1, 2, 4, 5, 8 or 9 (shown to have affinity for cells of the central nervous system). Administration may be via stereotactic surgery, intravenous administration following by various means of temporant disrupting the blood brain barrier described above.

What is claimed is:

1. A recombinant virus vector (RVV) comprising a virus and further comprising a nucleic acid encoding a neuroreceptor and/or a sub-peptide of a neuroreceptor.

2. The RVV of claim 1, wherein the nucleic acid encodes a 5-hydroxytryptamine receptor 4 (HTR4).

3. The RVV of claim 2, wherein the nucleic acid has the sequence set out as SEQ ID NO:1.

4. The RVV of claim 1, wherein the nucleic acid encodes a dopamine receptor D1(A).

5. The RVV of claim 4, wherein the nucleic acid has the sequence set out as SEQ ID NO:2.

6. The RVV of claim 1, wherein the nucleic acid encodes a GPR139.

7. The RVV of claim 6, wherein a nucleic acid has the sequence set at SEQ ID NO: 3.

8. The RVV of claim 1, wherein the viral vector is selected from groups consisting of gammaretroviruses, lentiviruses, flaviviruses, an influenza, an enterovirus, a rotavirus, a rubellavirus, a rubivirus, a morbillivirus, an orthopoxvirus, a varicellovirus, a dependoparvovirus, an alphabaculovirus, a betabaculovirus, a deltabaculovirus, a gammabaculovirus, a mastadenovirus, a simplexvirus, a varicellovirus, a cytomegalovirus, and combinations thereof.

9. The RVV of claim 1 wherein the viral vector is a lentivirus vector.

10. The RVV of claim 1 where the viral vector is an adeno-associated viral vector.

11. The nucleic acid of claim 2, wherein the nucleic acid encodes a linear sequential sub-peptide of HTR4 from positions 5 to 387 of SEQ ID NO: 1.

12. The gene insert of claim 4, wherein the nucleic acid encodes a linear sequential sub-peptide of D1(A) from positions 5 to 445 of SEQ ID NO: 2.

13. The gene insert of claim 6, wherein the nucleic acid encodes a linear sequential sub-peptides of GPR139 from positions 5 to 352 of SEQ ID NO: 3.

14. A method of treating a condition associated with the central nervous system, the method comprising a step of administering to a subject in need thereof a therapeutically effective amount of an RVV according to claim 1.

15. The method according to claim 14, wherein the viral vector is a lentivirus vector.

16. The method according to claim 14, wherein the viral vector is an adeno-associated viral vector.

17. The method according to claim 14, wherein the condition is selected from schizophrenia, addiction, depression, alcoholism, Parkinson's disease, psychosis, Post-Traumatic Stress Disorder (PTSD), and Alzheimer's disease.

18-24. (canceled)

25. The method according to claim 14, wherein the step of administration occurs by an intravenous route, an intramuscular route, an intraperitoneal route, an intrathecal route, an intravesical route, a topical route, an intranasal route, a transmucosal route, a pulmonary route, or a combination thereof.

26. The method according to claim 14, wherein the therapeutically effective amount is between about 10 to about 1×10¹⁶ TCID50/kg of the patient's body weight or is between about 10 to about 1×10¹⁶ TPC/kg of the gene vector.

27-28. (canceled)

29. The RVV of claim 10 wherein the adeno associated virus is selected from the group consisting of AAV-1, AAV-2, AAV-4, AAV-5, AAV-8 and AAV-9.

30. The RVV vector of claim 29 further comprising a nucleic acid encoding a GPCR.

31. The RVV vectors of claim 30 wherein the GPCR is GP139.


SEQUENCE LISTING (3 sequences)

SEQ ID NO: 1 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 2 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 3 - protein, 353 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP